(19) United States
(12) Patent Application Publication
(10) Pub. No.: US 2026/0027302 A1
(43) Pub. Date: Jan. 29, 2026

(54) INJECTION DEVICE

(71) Applicant: Owen Mumford Limited, Woodstock, Oxfordshire (GB)

(72) Inventor: Robert William BRUNS, Woodstock, Oxfordshire (GB)

(21) Appl. No.: 19/121,541

(22) PCT Filed: Oct. 17, 2023
(86) PCT No.: PCT/EP2023/078892

§ 371 Date: Apr. 16, 2025

(30) Foreign Application Priority Data
Oct. 17, 2022 (GB) 2215315.9

(51) Int. Cl. A61M 5/20; A61M 5/315

(52) U.S. Cl. CPC A61M 5/2033; A61M 5/31578; A61M 2005/2013; A61M 2005/206; A61M 2205/0216; A61M 2205/0238; A61M 2205/42; A61M 2205/8281

(57) ABSTRACT

An injection device comprising: a main body for receiving a syringe containing a medicament: and a drive mechanism within the main body and having a plunger driver movable through the main body. the drive mechanism comprising one or more drive springs that can be primed and released to move the plunger driver, the drive mechanism further comprising one or more damping elements of a resilient material for contacting the or each drive spring and thereby damp vibrations of the spring or springs following release.

### TECHNICAL FIELD

The present invention relates to an injection device for drug delivery provided with a noise damper of a resilient or viscous material.

### BACKGROUND

An injection device is a device for delivering a dose of drug from a medicament containing syringe or cartridge without the user having to manually apply motive force to drive a syringe plunger or cartridge bung. Injection devices may comprise injection devices designed to provide a single dose or, alternatively, pen devices where a user can set an amount of medicament to be provided. Typical injection devices such as autoinjectors and pen devices use powerful helical springs, which are primed and fired to provide this motive force.

### SUMMARY

According to a first aspect of the present invention, there is provided an injection device comprising: a main body for receiving a syringe containing a medicament; and a drive mechanism within the main body and having a plunger driver movable through the main body The drive mechanism comprises one or more drive springs that can be primed and released to move the plunger driver and one or more damping elements of a resilient material for contacting the or each drive spring to thereby damp vibrations of the spring or springs following release. Advantageously, the noise damping element is independent from any structure within the injection device other than the drive spring(s). This means that any vibrations which are created by the spring is not transmitted to another structure.

The one or more damping elements may be elongate members disposed within the or each drive spring. The outer transverse dimensions of the or each damping element may be smaller than the inner transverse diameter of the drive spring in which it is disposed so as to permit relative movement. The length of the or each damping element may be greater than the relaxed length of the drive spring in which it is disposed.

The one or more damping elements may be a cylinder, for example, a cylindrical rod or a cylindrical tube.

The injection device may further comprise a shuttle configured to travel along a shuttle guide in the main body of the injection device during priming. The shuttle guide being coupled to the plunger driver by the one or more drive springs. Prior to priming, the one or more drive springs are slightly tensioned so that adjacent turns of said springs are spaced apart. This can be achieved, for example, if prior to priming, the shuttle and plunger driver are in engagement and their combined length is configured to impart slight tensioning of the one or more drive springs.

The resilient material of the one or more damping element may be an elastomeric polymer, such as natural rubber, a synthetic rubber, or a mixture thereof.

The one or more damping elements may alternatively be provided as a sleeve through which the or each drive spring extends, or as a coating on the or each drive spring.

The one or more drive springs may be primed by a user action.

According to a second aspect of the present invention, there is provided an injection device comprising: a main body for receiving a syringe containing a medicament and a drive mechanism within the main body and having a plunger driver movable through the main body. The drive mechanism comprises one or more drive springs that can be primed and released to move the plunger driver. One or more of the drive springs are at least partially coated in a viscous material which damp vibrations of the spring or springs following their release.

### BRIEF DESCRIPTION OF THE DRAWINGS

FIG. 1A-C show an autoinjector in a (A) closed, (B) partially open and (C) open state;

FIG. 2 shows a safety syringe;

FIG. 3 shows a capped end of the autoinjector of FIGS. 1A-C;

FIGS. 4A and 4B show partial cross-sections of the autoinjector of FIGS. 1A-C;

FIG. 5 shows a shuttle, plunger driver and biasing element of the autoinjector of FIGS. 1A-C; and

FIGS. 6A and 6B show a biasing element and a noise damping element of the autoinjector of FIGS. 1A-C.

### DETAILED DESCRIPTION

Proposed is an autoinjector having a noise damping element coupled to a biasing element so that, after the biasing element is fired following priming, transverse and/or longitudinal vibrations which may develop, are damped.

The terms “forward” or “front” is used here refer to the needle side or injection site end of the autoinjector, whereas the term “rear” refers to the end of the autoinjector remote from the needle or injection site.

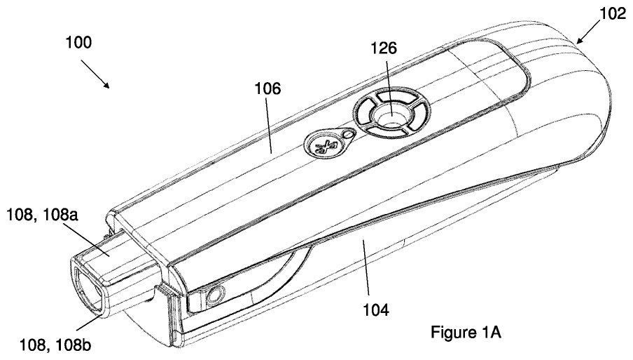

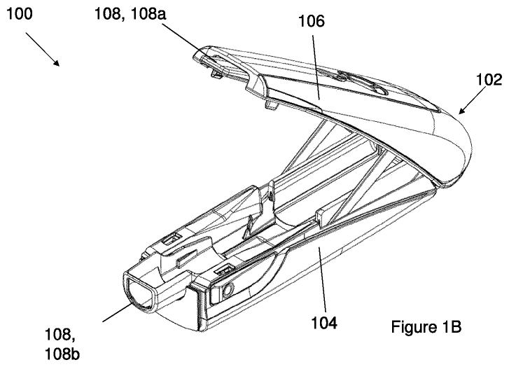

FIGS. 1A-C show an embodiment of an autoinjector 100 in: A) a closed state; B) a partially open state; and C) a fully open state.

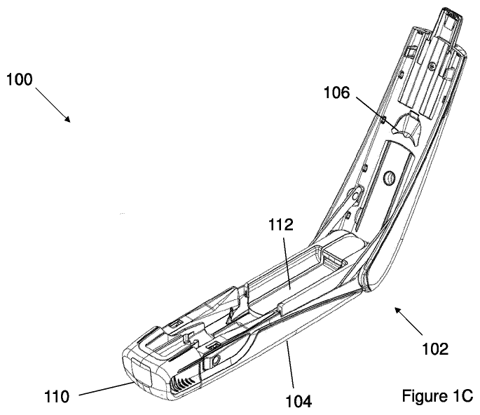

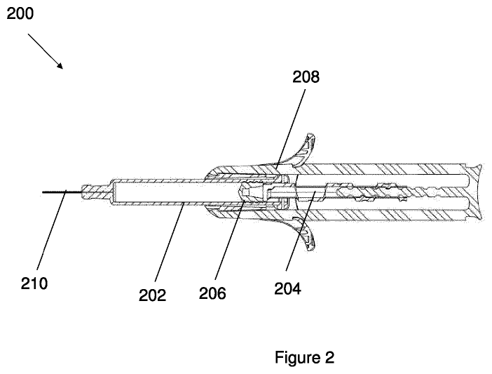

The autoinjector 100 comprises a housing 102 which includes a main body 104 and a lid 106 that are hingedly connected so as to permit opening and closing of the housing. The autoinjector further comprises a plurality of component parts contained within the housing. A syringe such as the syringe 200 of FIG. 2 (not shown in FIGS. 1A-C) is receivable within the housing in a slot 112 defined in the main body. The lid 106 of the autoinjector 100 includes a through-hole 126 positioned so that a surface of a plunger driver 116, the operation of which is described below, is viewable once firing is complete. That surface is vividly coloured in contrast to other parts visible through the through-hole prior to and during drug delivery to thereby provide a visual indication to the user that drug delivery is complete.

As shown most clearly in FIGS. 1A and 1B, the autoinjector 100 further comprises a shroud 108, formed from a lower part 108a and an upper part 108b. The lower and upper parts are respectively coupled to the main body 104 and lid 106 so that the parts 108a, 108b separate as the housing 102 is opened to allow insertion of a syringe 200 and come together to form the unitary shroud 108 when the housing is closed. The shroud 108 defines an aperture through which the needle 210 of a syringe 200 at least partially extends, when a syringe is received in the autoinjector 100. The lower part and upper part 108a, 108b comprise slidable connections with the main body 104 and lid 106 respectively, to permit movement between an extended position where the end of the syringe needle is substantially covered by the shroud, and a retracted position where the end of the syringe needle is exposed. The shroud parts are separately biased towards the extended position so that the needle of a syringe in the autoinjector remains substantially covered prior to an injection.

As shown in FIG. 1C, the autoinjector 100 includes a removable cap 110 which is normally in place prior to an injection being performed. For ease of understanding, the cap is omitted from FIG. 1A and 1B. In the configuration shown, the cap fits slidably over the lower shroud part 108b and further abuts against a front end of the main body 104. The cap 110 prevents user access to the shroud and hence accidental firing while the cap is in place.

FIG. 2 illustrates a safety syringe 200 suitable for use with the described autoinjector 100. Such a syringe 200 is described in detail in WO2019086718. It is sufficient here to note that the syringe comprises a syringe body 202 for containing a medicament, a syringe plunger 204 that engages with a bung 206 within the syringe body, a needle shield 208 coupled to the syringe plunger, and a needle 210. The coupling between the syringe plunger and the needle shield is such that the needle shield 208 is deployed around the needle of the syringe so as to substantially cover the needle following delivery of the medicament from the syringe body. This coupling is described in detail in WO2019/086718.

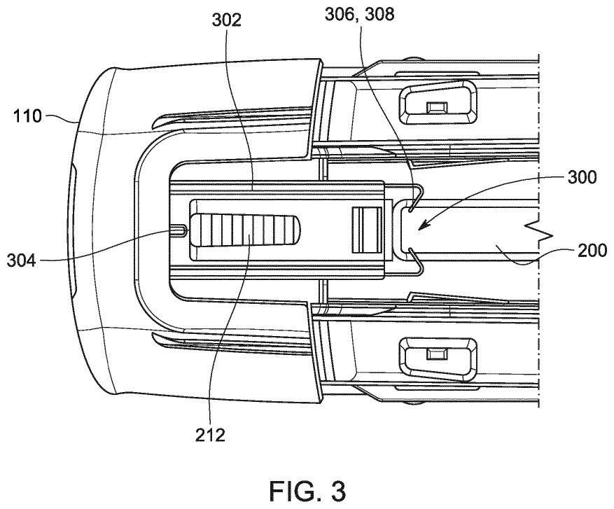

In general, syringes, including safety syringes, are routinely provided with a protective rigid needle shield (RNS) which require removal before a syringe can be used (the RNS is not shown in FIG. 2). To this end, the cap 110 also operates, in a known way, as an RNS remover 300. FIG. 3 shows a top plan view of the end of the autoinjector with the cap in place. The autoinjector 100 is in the open state such that the RNS remover and the end of a syringe 200 with an attached RNS 212 are visible. The RNS remover comprises a side wall 302 extending away from the cap, which defines a passageway 304 for receiving the RNS when the cap is fitted to the autoinjector. The front end of the side wall terminates with a gripping member 306. The gripping member is configured to allow easy insertion of the RNS 212 into the passageway whilst preventing its withdrawal thereafter. The RNS can therefore be removed from the syringe as the cap is removed.

In the configuration shown in FIG. 3, the gripping member 306 comprises projections 308 which extend inwardly into the passageway 304, angled away from the front end the side wall 302. As the syringe 200 with RNS is inserted into the slot 112 of the main body 104, the projections 308 are able to flex outwardly, whereas any return motion is prevented by the projections 308 as they come into engagement with the RNS 212.

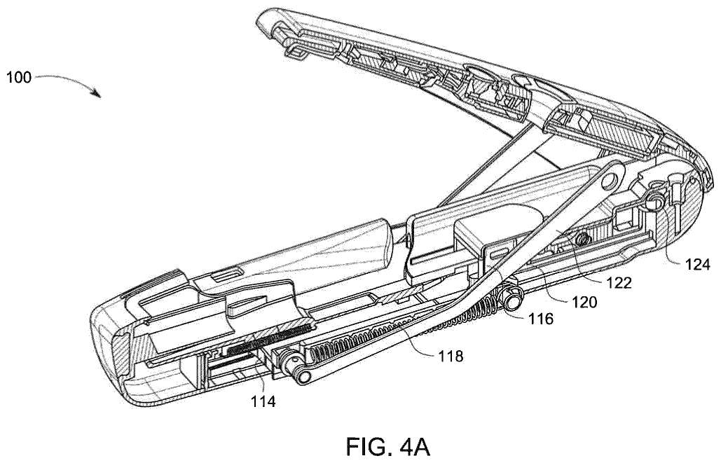

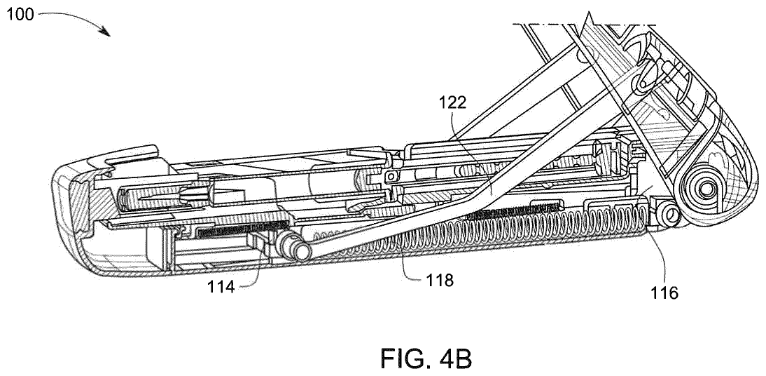

FIGS. 4A and 4B show partial cross-sectional views of the autoinjector 100 during various stages of priming to illustrate the presence and operation of further internal components during lid 106 opening and closing strokes. In particular, it can be seen that the autoinjector 100 comprises a shuttle 114 which is operable to move between a first 10 forward position and a second rearward position along a shuttle guide 120 on the main body 104 of the housing 102, a plunger driver 116 for driving the syringe plunger 204, and a biasing element 118 which couples the shuttle and plunger driver 116. The shuttle and plunger driver are slidably connected to the shuttle guide 120 to permit rearward and forward movement within the housing 102. Unlike the plunger driver, the shuttle is also fixedly connected to the lid 106 via two arm members 122.

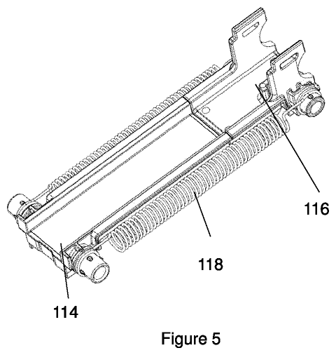

The assembled arrangement of the shuttle, plunger driver and biasing element prior to priming is shown in FIG. 5, i.e. this is the configuration of the arrangement in situ within the device and prior to priming. As shown, the biasing element 118 comprises two extension springs 118a, 118b which, prior to any priming, are under slight tension so as to hold the plunger driver 116 and shuttle 114 together. It should therefore be noted that priming, in the context of the extension springs, refers to the process of further tensioning the extension springs into a state whereupon firing can be initiated.

Each of the shuttle guide 120 and the plunger driver 116 comprise part of a latching arrangement, which are configured to cooperate to secure the plunger driver at the rear end of the autoinjector 100. A suitable latching arrangement is described in WO2022179832.

The autoinjector 100 further comprises a torsion spring 124 arranged at the hinged connection between the lid 106 and main body 104 of the autoinjector 100. The torsion spring is coupled to both the lid and main body. In the embodiment shown, one end of the torsion spring is attached to the lid, and the opposing end is attached to the main body of the autoinjector.

Priming of the autoinjector on the lid opening stroke (FIG. 4A) and the lid closing stroke (FIG. 4B) is now described. WO2021058474 describes operation of a similar autoinjector, except that the biasing element described therein further includes a compression spring.

As the lid 106 is opened, the arm members 122 which couple the lid and shuttle 114 together cause the shuttle to move rearwards from the first position to the second position. As shown most clearly in FIG. 5, the shuttle is in constant engagement with the plunger driver 116 so that its rearward travel causes the same rearward travel for the plunger driver. The extension springs 118a, 118b coupled between them therefore remains un-primed (i.e. further extended) during lid opening. Near the end of lid opening stroke, the latching arrangement part on the shuttle guide 120 and the plunger driver are brought together such that they are able to cooperate to secure the plunger driver at the rear end of the autoinjector 100.

Lid 106 opening also causes the end of the torsion spring 124 attached to the lid to rotate about its spring axis relative to the opposing end of the torsion spring. This primes the torsion spring on lid opening. When primed, the torsion spring produces a restoring force which tends to urge the lid closed.

Upon closing of the lid 106, whilst the shuttle 114 is free to move forwards along the shuttle guide 120 to the first position, the plunger driver 116 is held at the rear of the autoinjector by the latching arrangement. Thus, during the lid closing stroke, the shuttle and plunger driver separate and the extension springs 118a, 118b coupled between them are primed (i.e., further tensioned).

As has already been noted above, the primed torsion spring 124 urges the lid 106 closed. This assists in priming the extension springs 118 during closing, whilst requiring a minimal force to prime the torsion spring during opening. This is important for users of autoinjectors who would otherwise find it difficult to apply the necessary force to close the lid.

Firing of the autoinjector is now described. The firing mechanism is described in more detail in WO2022179832.

To fire the loaded and primed autoinjector, the user urges the front end of the autoinjector 100 into contact with an injection site (e.g., a user's skin). This causes the shroud parts 108a, 108b to move into the retracted position against their biases (e.g., respective springs). As the shroud retracts into the housing 102, the lower shroud 108b permits or causes release of the latching arrangement and the primed extension springs 118a, 118b. The restoring force of the extension springs, acting on the plunger driver 116, drives the plunger driver forwards to depress the syringe plunger and force the drug from out of the syringe needle into the injection site.

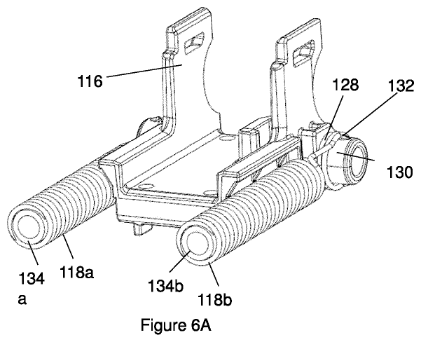

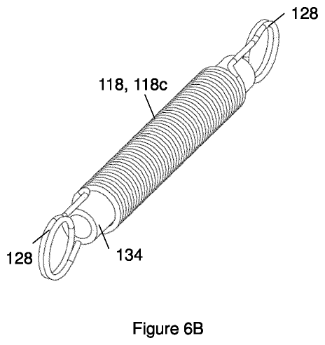

FIG. 6A shows a diagram of one end of the extension springs 118a, 118b coupled to the plunger driver 116. The extension springs have hooped ends 128 that can be fitted around an attachment point 130 on the plunger driver. The hooped ends are secured in place around the attachment point 130 by a tab 132. An equivalent coupling is provided at the shuttle 114. As has already been mentioned, release of the extension springs releases a large amount of energy which can, without mitigation, result in excessive noise that can unnerve the user. For this reason, it is proposed that a noise damping element 134 be coupled to the biasing element 118 in such a way that any transverse and/or longitudinal vibrations which may develop during, or immediately after, firing are damped.

FIG. 6B shows the noise damping element 134 and the extension spring 118 prior to assembly within the device, when the spring is in a relaxed state. As illustrated, the noise damping element 134 may take the form of a rubber (or other resilient material) cylinder 134a, 134b, two of which cylinders are inserted inside respective extension springs 118a, 118b. The diameters of the cylinders 134a, 134b are slightly smaller than the inner diameter of the extension springs 118a, 118b to permit movement within the springs. This prevents the cylinders from catching on the extension springs during firing. The cylinders are retained within the extension springs at either end by the shuttle 114 and plunger driver 116. The lengths of the cylinders are slightly greater than the lengths of the coiled regions 118c between the two hooped ends 128 of the extension springs, when in a relaxed state. In some examples, each cylinder is also greater in length than the coiled region of the extension spring when they are assembled in the device and the spring is in the unprimed state. Cylinders of these dimensions are particularly well suited to damp longitudinal and transverse vibrations.

Any transverse vibration that develops during firing (or otherwise) will cause the inner surface of the extension springs 118a, 118b to move into contact with the cylinders 134a, 134b. The cylinders restrict this transverse displacement, thereby reducing the amplitude and duration of any transverse vibration. Forming the cylinders of rubber, synthetic rubber or similar resilient material is effective at converting the kinetic energy of the extension spring into heat (without making a noise itself). Advantageously, the noise damping element is freestanding i.e. independent from any structure within the injection device other than the springs. This means that any vibrations which are created by the spring is not transmitted to another structure.

During firing, longitudinal vibrations primarily develop and propagate as adjacent turns in the central coiled region 118c rebound against one another. However, with the cylinders 134a, 134b inserted inside the extension springs, the cylinders have to be compressed before adjacent turns are able to contact one another. The compression of the cylinders dissipates energy, meaning that longitudinal vibrations that form from contact between adjacent turns are damped. In this way, the cylinders are able to damp longitudinal vibrations during firing.

Turning back to FIG. 5, it can be seen that the combined length of the shuttle 114 and plunger driver 116 is larger than the length of the central coiled region 118c of the extension springs 118a, 118b so that the extension springs are under tension even before their priming. This advantageously spaces apart adjacent turns in the extension springs to reduce coil rebounding upon firing. Moreover, as the extension springs are physically unable to enter a state of compression, resonant vibration modes that can arise from spring oscillations between a compressed and tensioned state are suppressed.

Although, the noise damping element 134 has been described as a hollow circular cylinder, other cross-sections and forms are possible. For example, it may be a rubber sleeve through which an extension spring extends or a rubber coating provided onto an extension spring. To avoid adjacent turns in the spring from sticking together, the rubber coating can be applied to the spring when it is in an extended state.

In a specific alternative embodiment, the extension springs 118a, 118b are at least partially coated in a viscous material (e.g., with a known smart grease). The coating can be applied manually or by dipping the springs into a vat containing the viscous material. The viscous material is such that it adheres to the springs with only very limited flow, whilst exhibiting a tendency to absorb mechanical energy during spring vibration which is dissipated as heat.

It is also envisaged that a plurality of noise damping elements could be inserted within each extension spring. The lengths of the damping element described above should then be interpreted as the total length of those damping elements. Other noise damping materials are also possible and envisaged by the skilled reader. For example: synthetic rubbers like neoprene, polymers such as silicone, and foams.

Any other biasing element described above (e.g., the torsion spring, the spring biasing the shroud 108 into the extended position) may also be fitted with a noise damping element.

Although the above invention has been described in relation to an autoinjector the skilled person will understand that it may also be applied to other injection devices such as pen devices where a user manually selects a size of the dose of medicament to be administered.

The skilled reader will be able to envisage further embodiments of the invention without departing from the scope of the appended claims.

1. An injection device comprising:
a main body for receiving a syringe containing a medicament; and
a drive mechanism within the main body and having a plunger driver movable through the main body, the drive mechanism comprising one or more drive springs that can be primed and released to move the plunger driver, the drive mechanism further comprising one or more damping elements of a resilient material, the damping elements being independent of the main body and contacting the or each drive spring to thereby damp vibrations of the spring or springs following release.

2. The injection device according to claim 1, wherein the one or more damping elements are elongate members disposed within the or each drive spring.

3. The injection device according to claim 2, wherein the outer transverse dimensions of the or each damping element are smaller than the inner transverse diameter of the drive spring in which it is disposed so as to permit relative movement.

4. The injection device according to claim 2, wherein the length of the or each damping element is greater than the relaxed length of the drive spring in which it is disposed.

5. The injection device according to claim 1, wherein each of the one or more damping elements is a cylinder.

6. The injection device according to claim 1, further comprising: a shuttle configured to travel along a shuttle guide in the main body of the injection device during priming and which is coupled to the plunger driver by the one or more drive springs, wherein, prior to priming, the one or more drive springs are slightly tensioned so that adjacent turns of said springs are spaced apart.

7. The injection device according to claim 6, wherein, prior to priming, the shuttle and plunger driver are in engagement and their combined length is configured to impart the slight tensioning of the one or more drive springs.

8. The injection device according to claim 1, wherein the resilient material of the one or more damping elements is an elastomeric polymer.

9. The injection device according to claim 8, wherein the elastomeric polymer is a natural rubber, a synthetic rubber, or a mixture thereof.

10. The injection device according to claim 1, wherein the one or more damping elements are provided as a sleeve through which the or each drive spring extends.

11. The injection device according to claim 1, wherein the one or more damping elements are provided as a coating on the or each drive spring.

12. The injection device according to claim 1, wherein the one or more drive springs can be primed by a user action.

13. An injection device comprising:
a main body for receiving a syringe containing a medicament; and a drive mechanism within the main body and having a plunger driver movable through the main body, the drive mechanism comprising one or more drive springs that can be primed and released to move the plunger driver, wherein, the one or more drive springs are at least partially coated in a viscous material which damps vibrations of the spring or springs following release.

14. The injection device according to claim 13, further comprising:
a shuttle configured to travel along a shuttle guide in the main body of the injection device during priming and which is coupled to the plunger driver by the one or more drive springs, wherein, prior to priming, the one or more drive springs are slightly tensioned so that adjacent turns of said springs are spaced apart.

15. The injection device according to claim 14, wherein, prior to priming, the shuttle and plunger driver are in engagement and their combined length is configured to impart the slight tensioning of the one or more drive springs.

16. The injection device according to claim 13, wherein the one or more drive springs can be primed by a user action.
